(19) United States
(12) Patent Application Publication
(10) Pub. No.: US 2025/0095814 A1
(43) Pub. Date: Mar. 20, 2025

(54) DETERMINING AND PERFORMING OPTIMAL ACTIONS ON A SYSTEM

(71) Applicant: Microsoft Technology Licensing, LLC, Redmond, WA (US)

(72) Inventors: Chao MA, Cambridge (GB); Jiaqi ZHANG, Cambridge (GB); Cheng ZHANG, Cambridge (GB); Joel JENNINGS, Cambridge (GB); Colleen TYLER, Redmond, WA (US); Marife DEFANTE, Woodinville, WA (US); Lisa Lynne PARKS, Seattle, WA (US)

(21) Appl. No.: 18/537,794

(22) Filed: Dec. 12, 2023

Related U.S. Application Data
(60) Provisional application No. 63/584,101, filed on Sep. 20, 2023.

(51) Int. Cl. G16H 20/00; G06N 5/046

(52) U.S. Cl. CPC G16H 20/00; G06N 5/046

(57) ABSTRACT

In certain examples, a causal inference model is trained on a re-balancing task in a self-supervised manner, using ‘unlabelled’ training data pertaining to multiple domains. Rather than approaching casual inference as a domain-specific task (e.g., designing one causal-inference approach for a particular manufacturing application, another for a particular aerospace application, another for a specific medical application etc.,) a general-purpose causal inference mechanism is learned from a large, diverse training set that contains many treatments dataset over many field/applications (e.g., combining manufacturing data, engineering data, medical data etc. in a single dataset used to train a single neural network). In other words, a cross-domain causal inference model is trained, which can then be applied to a dataset in any domain, including domains that were not explicitly encountered by the neural network during training.

### CROSS-REFERENCE TO RELATED APPLICATION

This application claims priority to U.S. Provisional Patent Application No. 63/584,101, entitled “DETERMINING AND PERFORMING OPTIMAL ACTIONS ON A PHYSICAL SYSTEM,” filed on Sep. 20, 2023, the disclosure of which is incorporated herein by reference in its entirety.

### TECHNICAL FIELD

The present disclosure relates to methods and systems for determining and performing optimal actions on a system.

### BACKGROUND

Causal inference is a fundamental problem with wide ranging real-world applications in fields such as manufacturing, engineering and medicine. Causal inference involves estimating a treatment effect of actions on a system (such as interventions or decisions affecting the system). This is particularly important for real-world decision makers, not only to measuring the effect of actions, but also to pick the best action that is the most effective.

For example, in the manufacturing industry, causal inference can help quantitatively identify the impact of different factors that affect product quality, production efficiency, and machinery performance in manufacturing processes. By understanding causal relationships between these factors, manufacturers can optimize their processes, reduce waste, and improve overall efficiency. As another example, in the field of engineering, causal inference can be used for root cause analysis and identify underlying causes of faults and malfunctions in machines or electronic systems such as vehicles or unmanned drones (e.g. aircraft systems). By analyzing data from sensors, maintenance records, and incident reports, causal inference methods can help determine which factors are responsible for observed issues and guide targeted maintenance and repair actions. In genome-wide association studies (GWAS), causal inference may be used, for example, to associate between genetic variants and a trait or disease, accounting for potential confounding factors, which in turn may allow therapeutic treatments to be developed or refined.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Nor is the claimed subject matter limited to implementations that solve any or all of the disadvantages noted herein.

Example embodiments herein provide a “causally-aware” foundation model and training method. The causally-aware foundation model is able to identify and quantify underlying causal effects. In certain examples, a neural network or other causal inference model is trained on a re-balancing task in a self-supervised manner, using ‘unlabelled’ training data pertaining to multiple (e.g., many) domains (e.g., different fields, applications and/or use cases). Rather than approaching casual inference as a domain-specific task (e.g., designing one causal-inference approach for a particular manufacturing application, another for a particular aerospace application, another for a specific medical application etc.,) a general-purpose causal inference mechanism is learned from a large, diverse training set that contains many treatments dataset over many field/applications (e.g., combining manufacturing data, engineering data, medical data etc. in a single dataset used to train a single neural network). In other words, a cross-domain causal inference model is trained, which can then be applied to a dataset in any domain, including domains that were not explicitly encountered by the neural network during training.

### BRIEF DESCRIPTION OF FIGURES

Illustrative embodiments will now be described, by way of example only, with reference to the following schematic figures, in which:

FIG. 1 shows an example observational treatment dataset on which causal inference is performed.

FIG. 2 shows a schematic block diagram of independent domain specific causal inference models and respective training sets.

FIG. 3 shows a schematic function block diagram of the causal inference model as configured in a training stage.

FIG. 4A shows a block diagram of a forward mode training setup for a causal inference model with a single dataset setting.

FIG. 4B shows the forward diagram of a forward mode training setup for a causal inference model with a multi-dataset setup.

FIG. 5A shows a schematic function block diagram of a trained causal inference model during inference time.

FIG. 5B shows a schematic block diagram of a treatment effect estimation function on a new dataset using a trained causal inference model.

FIG. 6 shows an example of a training architecture for a causal inference.

FIG. 7 shows an example architecture of a trained causal inference model.

FIG. 8 shows a non-limiting example of a computing system.

### DETAILED DESCRIPTION

Particular embodiments will now be described, by way of example only.

Causal inference has numerous real-world applications. Causal inference may interface with the real-world in term of both its inputs and its outputs/effects. For example, multiple candidate actions may be evaluated via causal inference, in order to select an action (or subset of actions) of highest estimated effectiveness, and perform the selected action on a physical system(s) resulting in a tangible, real-world outcome. Input may take the form of measurable physical quantities such as energy, material properties, processing, usage of memory/storage resources in a computer system, therapeutic effect etc. Such quantities may, for example, be measured directly using a sensor system or estimated from measurements of another physical quantity or quantities. Logical systems implemented within physical systems are also considered, such as software systems implemented on a physical computer system. Actions performed at the level of a logical system will ultimately result in physical effects (such as the movement of a file or the sending of a message within a distributed computer system in response to some action).

For example, different energy management actions may be evaluated in a manufacturing or engineering context, or more generally in respect of some energy-consuming system, to estimate their effectiveness in terms of energy saving, as a way to reduce energy consumption of the energy-consuming system. A similar approach may be used to evaluate effectiveness of an action on a resource-consuming physical system with respect to any measurable resource.

A ‘treatment’ refers to an action performed on a physical system. Testing may be performed on a number of ‘units’ to estimate effectiveness of a given treatment, where a unit refers to a physical system in a configuration that is characterized by one or more measurable quantities (referred to as ‘covariates’). Different units may be different physical systems, or the same physical system but in different configurations characterized by different (sets of) covariates. Treatment effectiveness is evaluated in terms of a measured ‘outcome’ (such as resource consumption. Outcomes are measured in respect of units where treatment is varied across the units. For example, in one a ‘binary’ treatment set up, a first subset of units (the ‘treatment group’) receives a given treatment, whilst a second subset of units (the ‘control group’) receives no treatment, and outcomes are measured for both). More generally, units may be separated into any number of test groups, with treatment varied between the test groups.

A challenge when evaluating effectiveness of actions is separating causality from mere correlation. Correlation arises from ‘confounders’, which are variables that can create a misleading or spurious association between two or more other variables. When confounders are not properly accounted for, their presence can lead to incorrect conclusions about causality.

One approach to this issue involves randomized experiments, such as A/B testing (also known as randomized control trials). With this approach, units subject to testing are randomly assigned to different variations, as a way to reduce bias. A/B testing attempts to address the issue of confounders by attempting to give even representation to confounders across the different test groups. In principle, this does not require confounders to be explicitly identified, provided the test groups are truly randomized.

In reality, truly randomized A/B testing may be challenging to implement in practice. Firstly, this approach generally requires an experiment designer to have control over the allocation of units to test groups, to ensure allocations are randomized. Moreover, even when an experiment designer has such control, it is often challenging to ensure truly randomized allocation.

There are three common methods to identify causal effects, including i) randomized experiments (A/B testing): ii) expert knowledge/existing knowledge: iii) observational study (building quantitative causal models solely based on non-experimental data). However, these methods lack flexibility, and are often not be feasible in practice due to either costs (too expensive to experiment), feasibility (not enough domain knowledge or non-experimental data) and/or ethical reasons, etc. Moreover, these methods/models are highly scenario-specific, for instance the causal model built for performing GWAS task cannot be re-used for the purpose of root causal analysis in aerospace industry.

Herein, an alternative method is described that addresses these problems, namely a “causal foundation model” for causal inference.

Foundation models such as language foundation models (e.g., large language models, such as GPT models) and image foundation models (e.g., DALL-E) have been built. However, in contrast to existing foundational models, a foundational paradigm is provided herein for building general-purpose machine learning systems for causal analysis, in which a single model trained on a large amount of unlabeled data can be adapted to many applications in causal inference across different domains. In other words, a single machine learning model is built that, once trained, can be directly used in any domain for any problem that can be characterized as “estimating effects of certain actions from data”. It can be instantly used in manufacturing industry, scientific discovery, medical research, aerospace industry etc. with none or little adjustment. The approach herein not only yields a significant saving in costs and resources compared to a conventional approach, which would require development of solutions in those scenarios specifically, but does so while achieving similar or even better performance. The present approach ultimately utilizes computer resources more efficiently than conventional methods because, once a generally applicable causal inference model has been trained, it can be readily applied to other domains without further training. Therefore, over a large number of domains, a significant reduction in computer resources required for causal model training is achieved.

Recent advances in artificial intelligence has witnessed the trend of paradigm shift with models trained on broad data that can be adapted to different tasks. Such models have been characterized as foundation models. These models, often trained using self-supervision, are able to induce knowledge embedded in data of different forms, including natural language, images, and biological sequencings. This acquired knowledge is helpful when the model is asked to perform tasks in novel scenarios. With vast amounts of data becoming increasingly available from diverse sources, such models can be of interest to leverage information that can be learned in order to build more intelligent systems.

Intelligent systems, aside from leveraging information embedded in data, should also learn about the underlying mechanisms that drive cause-and-effect relationships, which has been recognized. As one desired property of these systems can be better decision-makings, understanding causality is vital to make accurate predictions across various domains including healthcare, economics and statistics. It has been shown that models based only correlation could be subject to spurious correlations. For example, it was observed that between 2000 to 2009 per capita cheese consumption is highly correlated with number of people who were entangled with their bedsheet. Without understanding causality and relying solely on correlation, a cheese factory that would like to modify their sales rate might erroneously think it is related to bedsheet qualities.

Previous works laid out the foundations for causal inference with statistical guarantees. However, by leveraging large-scale data collection, a “causal” foundation model is provided herein that is significantly more scalable than conventional methods. Various practical challenges in building such models are addressed herein. Unlike text and images, which can be relatively well-structured, common datasets used in causality, e.g., for treatment effect estimation, contain complex relationships between variables that might be heterogenous across datasets. These less-structured heterogenous relationships make it harder for the model to capture compared to linguistic or perceptual patterns. On top of this, the challenge also lies in how to create a suitable self-supervised task, which is an important consideration to build foundation models. For example, masked prediction of covariates may be insufficient when it captures independent features of a unit.

Foundation models have brought changes to the paradigm of machine learning which gives spark of human level intelligence across a large range of different tasks. However, a gap on complex tasks such as causal inference still remains due to challenges involving complex reasoning steps and high numerical precision requirement. In embodiments presented herein, a first step is taken towards building causally-aware foundation model for complex tasks. In particular, a new theoretically sound approach, naming causal inference with attention (CInA), that utilizes multiple unlabeled datasets and can be used to perform zero-shot causal reasoning on unseen tasks with new data is proposed. This is based on results unveiling the primal-dual connection between optimal balancing and self-attention, which enables zero-shot causal inference via the last layer of a trained transformer-type architecture. It is empirically demonstrated that this approach CInA can generalize well to out of distribution datasets and various different real-world datasets, reaching and even out-performing traditional per dataset causal inference approaches.

Conventional foundation models such as language foundation models and image foundation models may be powerful in terms of generating vivid images and human-like conversations, but they are not “causally-aware”, meaning that they cannot be used to estimate the underlying causal effects. Therefore, they are mostly purely “brute force algorithms” which makes them prone to issues such as hallucinations (generating plausible but incorrect outputs). On the contrary, embodiments herein provide a “causally-aware” foundation model that, despite being trained on non-experimental observational data, can still identify and quantify underlying causal effects without requiring of performing additional A/B experiments or expert knowledge. The causal foundational model can even on another task/domain which it has not encountered in training.

One embodiment described herein provides a method that learns how to estimate treatment effects on multiple datasets in an “end-to-end” fashion. End-to-end learning is a training technique in which the model learns all steps between an initial input phase and a final output phase (rather than training individual components separately). The specific end-to-end implementation described herein is powerful in its flexibility to incorporate different architectures and generalize to perform direct inference on new unseen datasets.

The method involves balancing covariates as a self-supervised task to learn treatment effects on multiple heterogenous datasets that may have arisen from various sources. By using the connection between optimal balancing and self-attention, the method shows how to solve optimal balancing via training models with self-attention as a final layer.

It is demonstrated herein that this procedure is guaranteed to find the optimal balancing weights on a single dataset under certain regularities, by using a primal-dual reasoning.

Empirically, it is demonstrated herein that this approach can generalize well to out of distribution datasets and various different real-world datasets, reaching and even out-performing traditional per dataset causal inference approaches.

An analysis is described herein, which motivates a concrete transformer architecture that can be exactly mapped to solutions of a Riesz representator (RR) learning problem. Those RR solutions can be directly used to perform causal inference with only non-experimental observational data. One example of such RR problem is the classical support vector machine (SVM) learning problem. In other words, to implement a causal foundational model, a transformer is trained to serve as a one-shot solver for any Riesz representator problems. Once trained, given observational data from any task or domain, it will directly predict the solutions of Riesz representator problem (without actually having to incur the computational expense of solving it), and use the predicted solutions to estimate the causal effects or any decision queries.

One such approach described herein may be used to estimate casual effect from imperfect, non-randomized datasets. The described approach can recognize and correct bias in any treatment dataset with N units of the form D={(Xᵢ, Tᵢ, Yᵢ)}_{i∈[N]}, where Xᵢ denotes a set of D observed covariates (where D is one or more) of the ith unit, Tᵢ denotes a treatment observation for the ith unit (e.g. an indication of whether or not a given treatment was applied to that unit), and Yᵢ denotes an outcome observed in respect of the ith unit. In the following X denotes an N×D matrix of covariates across the N units (where N is one or greater), T denotes an N-dimensional treatment vectors containing the treatment observations across the N units and Y denotes an N-dimensional vector of the N observed outcomes.

A ‘covariate balancing’ mechanism is used to account for biases exhibited in a dataset of the above form. Balancing weights are calculated and applied to the dataset, in order to reduce confounder bias, and thereby enable a more accurate estimation of casual treatment effect (that is, truly causal relationships between treatments and outcomes, as opposed to mere correlations between treatments and outcomes exhibited in the dataset). This, in turn, reduces the risk of selecting and applying sub-optimal treatments in the real-world.

In the described approach, a neural network is trained to generate a set of balancing weights α from a set of inputs. Whilst a neural network is described, the description applies equally to other forms of machine learning components. At inference, balancing weights α computed from a given dataset may then be used to rebalance the outcomes as αY.

A novel training mechanism is described herein, in which a neural network is trained on a covariate re-balancing task in a self-supervised manner, using large amounts of ‘unlabelled’ training data pertaining to many different domains (e.g., fields, applications and use cases). Rather than approaching casual inference as a domain-specific task (e.g. designing one causal-inference approach for a particular manufacturing application, another for a particular aerospace application, another for a specific medical application etc.,) a general-purpose causal inference mechanism is learned from a large, diverse training set that contains many treatments dataset over many field/applications (e.g. combining manufacturing data, engineering data, medical data etc. in a single dataset used to train a single neural network). In other words, a cross-domain causal inference model is trained, which can then be applied to a treatment dataset in any domain (including domains that were not explicitly encountered by the neural network during training).

In one approach, the balancing weights are generated from X and T provided as inputs to the neural network. In this approach, outcomes Y are not required to generate the balancing weights α. This, in turn, means it is not necessary to expose the neural network to outcomes during training. and it is therefore possible to train the neural network on datasets of the form {{X, T}ᵢ}, implying that the covariates are known and the assignment to treatment groups is known, but the outcomes may or may not be known). Here, the index j denotes the jth dataset belonging to the training set, where j=1 might for example be an engineering dataset, j=2 might be a manufacturing dataset, j=3 might be a medical dataset etc. The neural network may be conveniently denoted as a function ƒ_{θ} (X,T) where θ denotes parameters (such as weights) of the neural network that are learned in training. In the described architecture, the neural network returns an N-dimensional vector V as output—that is, ƒ_{θ} (X,T)=V—and rebalancing weights are computed from V as VT/Z

$$( implying {α}_{i} = \frac{{V}_{i} {T}_{i}}{Z i} for each i ) ,$$

where Z=h(X) is a renormalization factor computed as a function of the covariates X computed within the neural network. The parameters θ are learned in a self-supervised manner, from X and T alone (and, in this sense the training set {{X, T}ⱼ} is said to be unlabelled).

The neural network may be a “large” model, also referred to as a “foundational” model. Large models have typically of the order a billion parameters or more, trained on vast datasets. In the present context, a “causal foundational model” may be trained using the techniques described herein to be able to rebalance any treatment dataset, including treatment datasets relating to contexts, applications, fields etc that were not encountered during training

Training on examples of {X,T} without outcomes Y is viable because the training is constructed in a manner that causes the neural network to consider all possible outcomes, and minimize worst case scenario. This property makes the neural net generalizable and robust to any scenarios.

In another embodiments, outcome Y may be additionally incorporated into the training process. In this case, Y is also provided as input to the neural network. If the model is trained on synthetic and/or real datasets where treatment effects (ATE) are known, then the treatment effects ATE may be used as ground truth to compute a supervised signal. In other words, the training dataset now becomes D={(X, T, Y, ATE)}. During training, the neural network uses both a forward model and a test mode to produce both predictions for treatment vector and the ATEs, and an error is minimized for both the treatment vector (T) and the ATE.

In embodiments, a computer-implemented training method comprises training, refining, and accessing a machine learning (ML) causal inference model (such as a large ML model). The causal inference model can learn to solve arbitrary causal inference problems and decision-making problems using observational data from multiple (any) domains. Once trained on multiple data sources, the causal inference model is able to generalize to solve any tasks beyond training data. That is, the user may input a new data set, comprising observational records of any system of interest (in any domain): then the model can estimate a causal treatment effect of a selected treatment variable on any target variables. Based on the estimated causal effects, a system incorporating the causal inference model can recommend optimal actions to achieve optimal outcomes, or even perform such actions (or cause them to be performed) autonomously.

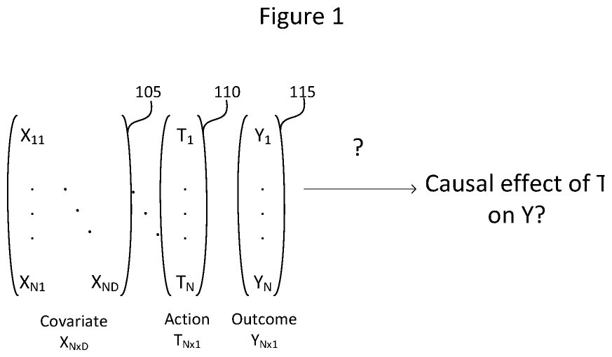

FIG. 1 shows an example treatment dataset on which causal inference may be performed. Treatments 110 are applied to covariates 105 to generate outcomes 115. Xᵢ represents an i-th matrix element of the covariate matrix 105. The covariate matrix 105 has dimensions N by D. The covariates 105 represent the entities to which treatments 110 are applied in a physical system. For example, the elements of the covariate matrix, covariates 105, can represent raw materials quality, machinery maintenance and process control in a manufacturing context.

Tᵢ represents an i-th element of a treatment vector 110. The treatment vector 110 has dimensions of N by 1. The number of treatments 110 corresponds to the number of entities within the system. In the manufacturing example, the example treatments 110 can represent energy management such that Tᵢ=1 implies active energy management is applied to the ith entity, and Tⱼ=−1 implies such treatment is not applied to the jth entity.

Similarly, Yᵢ represents an i-th element of the outcome vector 115. The outcome vector 115 has dimensions of N by 1. The dimensions of the outcome vector 115 are the same as that of the treatment vector 110. The outcomes 115 generated in the manufacturing example could for example be production efficiency.

The causal inference problem involves estimating causal effects of the treatments 110 on the outcomes 115 having applied the treatments 110 to the covariates 105, as shown in FIG. 1. For example, the aim might be to assess whether active energy management (an example treatment) actually has any causal effect on production efficiency (an example outcome). It is important in this context to reduce the influence of mere correlation on any conclusion drawn (a particular treatment dataset might indicate a correlation between production efficiency and active energy management, but in a biased dataset, that correlation may be non-causal).

The problem is solved using a causal inference model presented herein.

In a domain-specific approach, domain-specific causal inference models may be separately trained (e.g. to perform domain-specific covariate rebalancing). However, this approach lacks flexibility, resulting in models that cannot be applied to domains that have not been explicitly trained on, and also model inefficiency, as multiple models need to be trained an implemented, requiring an amount of computing and memory/storage resources that increases with number of domains of interest and the number of domain specific models.

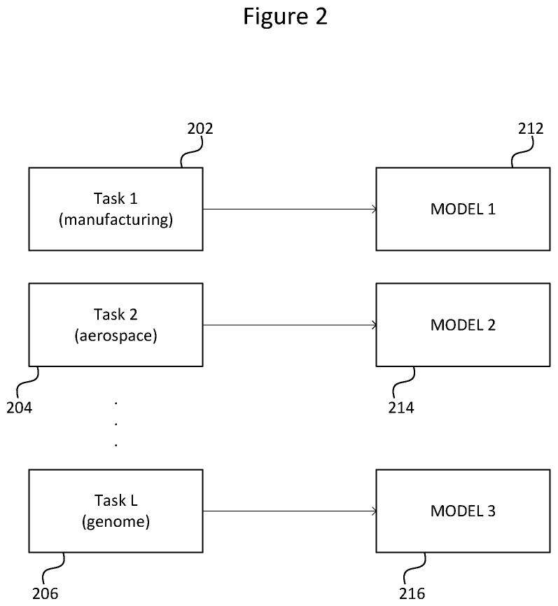

FIG. 2 shows an approach to determining causal effects according to domain specific models. The models in the figure represent causal inference models trained on specific datasets relating to the industry in which the models are used. For example Task 1 202 relates to model 1, 212, such that model 1, 212, has been trained on datasets relating to the manufacturing industry. Similarly, task 2 204 relates to model 2 214 such that model 2 214 has been trained on datasets relating to the aerospace industry. Task L 206 relates to model 3 216, such that model 3 216 has been trained on datasets relating to genomes. There can exist any number of models 212, 214, 216 that have all been trained separately on different datasets.

The domain specific models that are trained using the method described with reference to FIG. 2 are unable to find causal effects for datasets within domains that differ from the domain in which they have been trained. For example, model 1 212 would be unable to find a causal effect if the dataset from task 2 204 is received as input, or to tasks from another domain not encountered during training of any of the models. Hence, the set of domain-specific models cannot be readily extended to domains not encountered explicitly in training.

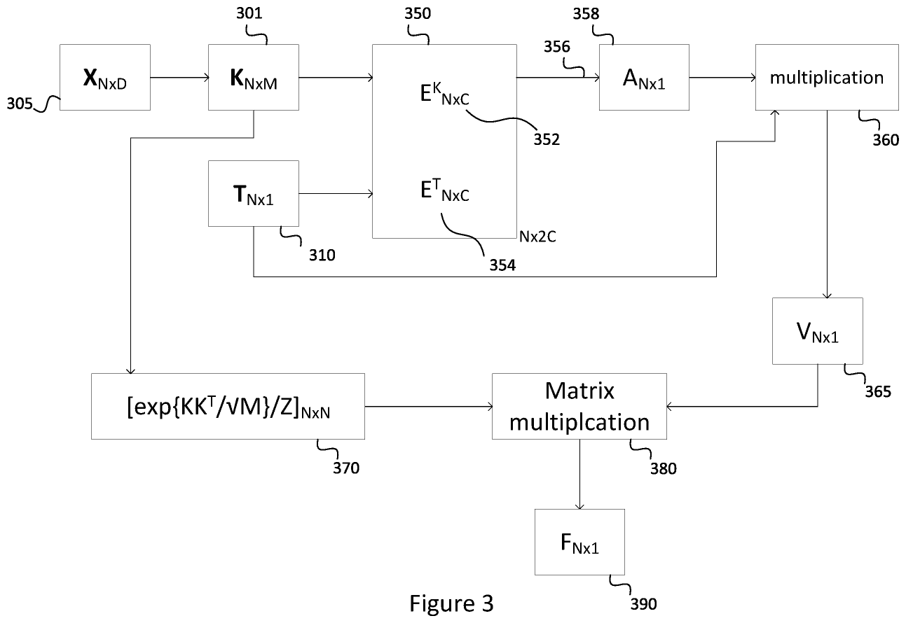

FIG. 3 shows a training method of one embodiment of a general purpose causal inference model.

In training, the causal inference model learns a row-wise embedding, that maps the covariates X 305 and treatments T 310 of each dataset Dᵢ to an embedding. The covariates are mapped to a key embedding K 301 (of size N by M, where M is the embedding size) that summarizes the row-wise information of the dataset.

The key embedding 301 and treatments 310 are then mapped to embedding E^{K} 352 and E^{T} 354 (of size N by C, where C is the embedding size) respectively. This embedding is called row-wise, since each row of E only depend on each row of the key embedding K 301 and treatments T 310. In one embodiment, such embedding is implemented by a neural network. The two embeddings E^{K} and E^{T} form a matrix 350 of size N by 2 C.

A self-attention layer 356 is shown in FIG. 3 and used to generate an attention vector A 358 of size N by 1.

Attention-based neural networks are a powerful tool for ‘general-purpose’ machine learning. Attention mechanisms were historically used in ‘sequence2sequence’ networks (such as Recurrent Neural Networks). Such networks receive sequenced inputs and process those inputs sequentially. Historically, such networks were mainly used in natural language processing (NLP), such as text processing or text generation. Attention mechanisms were developed to address the ‘forgetfulness’ problem in such networks (the tendency of such network to forget relevant context from earlier parts of a sequence as the sequence is processed: as a consequence, in a situation where an earlier part of the sequence is relevant to a later part, the performance of such networks tends to worsen as the distance between the earlier part and the later part increases). More recently, encoder-decoder neural networks, such as transformers, have been developed based solely or primarily on attention mechanisms, removing or reducing the need for more complex convolutional and recurrent architectures.

On top of the row-wise embedding E, the causal inference model learns a dataset-wise embedding, that maps Eᵢ of each dataset Dᵢ to a vector Vᵢ of size N by 1. In practice, such embedding may be implemented by a self-attention neural network layer or layers. This is followed by an ReLu activation function mapping and an element-wise multiplication 360 with the treatment vector, Tᵢ, 310 to generate the value vector V 365. The value vector 365 is of size N by 1.

For each dataset Dᵢ, the causal inference model simulates a forward mode output of the model, denoted by F 390, a vector of size N by 1, which is given by the matrix multiplication 380 between a softmax-kernel 370 of K 301, and the value vector V 365.

The softmax kernel 370 is a matrix of size N by N. It is calculated using an exponential of the key embedding 301 multiplied by its transpose and divided by the size of the key embedding, M. The exponential functions is then divided by a normalisation factor, Z.

Finally, the causal inference model is trained with the goal of driving the simulated forward mode outputs F 390 to be as close to the real observed treatment vectors T 310 in each of the aforementioned datasets D₁, D₂, . . . , D_{L} as possible.

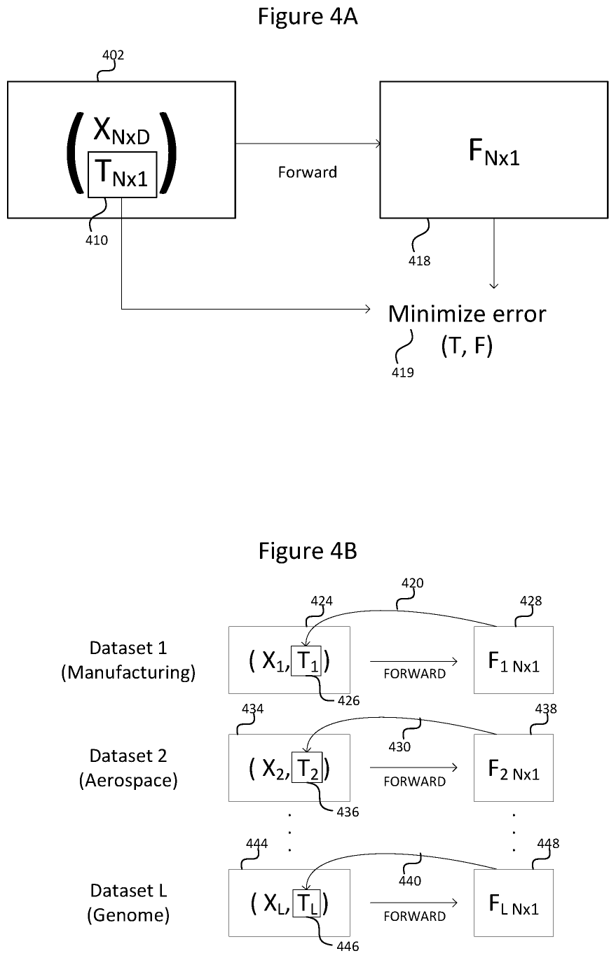

FIG. 4A shows a forward mode training of a causal inference model for a single dataset according to the present disclosure. A covariate matrix of size N by D and a treatment vector 410 of size N by 1 are shown in a single dataset 402. The training process of the causal inference model described with reference to FIG. 3 completes a forward pass to generate a simulated forward mode output 418. The simulated forward mode output 418 and the treatment vector 410 are input into a minimisation function 419. The function 419 aims to minimise the error between the treatment vector 410 and the simulated forward mode output 418.

FIG. 4B shows the forward mode training of a causal inference model for a multi-dataset according to the present disclosure. The model is trained on a set of multiple datasets (a training dataset of datasets), denoted by D₁, D₂, . . . , D_{L}, in so-called “forward” mode, with the goal of being able to simulate realistic synthetic samples that are as close to the provided multiple datasets as possible. To fully describe the logics, each dataset Dᵢ (1≤I≤L) may comprise three matrices, namely the covariates X (a matrix of size N by D, N and D might defer from datasets), the treatments T (a vector of size N by 1), and the outcome Y (a vector of size N by 1). However, as noted, the outcomes Y are not essential for training and are therefore omitted from this figure.

Each dataset is used to train the same causal inference model using the method described with reference to FIGS. 3 and 4A. The covariates and the treatments for each dataset are input into the model for training. The model then outputs a simulated forward mode output for each of the training datasets. The result is then backpropagated through the model to adjust the learned parameters or weights.

In FIG. 4B, dataset 1, 424, relating to manufacturing, is input into the causal inference model. The model performs a forward pass to generate a simulated forward mode output 428. This apples similarly to dataset 2, 434, up to some dataset L, 444, for any given domain. Example dataset 2 434, relating to aerospace, is input into the causal inference model which performs a forward pass to generate a simulated forward mode output 438. Additionally, dataset L 444, relating to genome, is input into the causal inference model which performs a forward pass to generate a simulated forward mode output 448.

The causal inference model performs a backward propagations 420, 430, 440 for each dataset to train the model by minimising the error between the treatment vectors 426, 436, 446 and the simulated forward mode outputs 428, 438, 448.

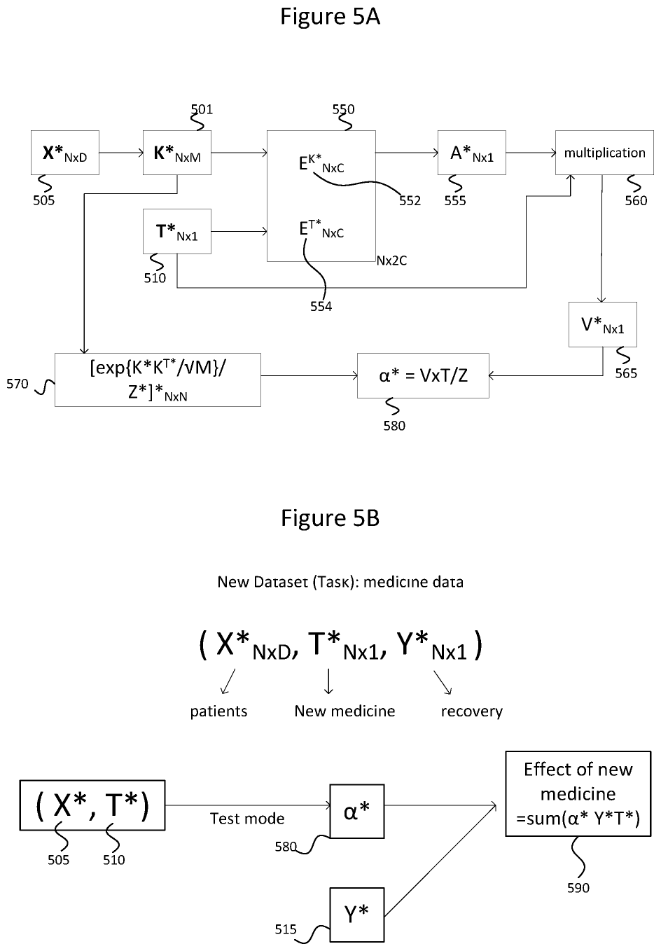

FIG. 5A shows the causal inference model after training. The causal inference model is used to determine a rebalancing weight, α*.

After training, the causal inference model may be used to estimate a target variable from among the variables of any given new dataset D* comprising N* data points, usually unseen by the model during training. This process involves estimating, given covariates X* 505 and treatments T* 510 of a new dataset D*, a corresponding value vector V*, 565, using forward mode.

Forward mode has been described with reference to FIG. 3 in training of the causal inference model however it is now performed with an unseen dataset as input. The covariates 505 are mapped to a key embedding K 501 (of size N by M, where M is the embedding size) that summarizes the row-wise information of the dataset.

The key embedding 501 and treatments 510 are mapped to embedding E^{K} 552 and E^{T} 554 (of size N by C, where C is the embedding size) respectively. This embedding is called row-wise, since each row of E only depend on each row of the key embedding K 501 and treatments T 510. In one embodiment, such embedding is implemented by a neural network. The two embeddings E^{K} and E^{T} form a matrix 550 of size N by 2 C.

A self-attention layer 556 is shown and used to generate an attention vector A 358 of size N by 1. The attention vector 555 and the treatments 510 perform a matrix multiplication 560 to generate the value vector 565.

Causal balancing weights α* 580, a of size N* by 1, are generated by first multiplying 560 V* 565 by T* 510, then renormalizing the values with a certain renormalization factor, Z*. The normalization factor arising from the softmax kernel 570.

A causal treatment effect of the variable T* 510 on Y* is calculated by first multiplying the balancing weights α*, 580, treatments T* 510, and outcomes Y*, and then finally summing up all the values obtained in the said multiplication. This process is described in more detail below.

### An Adversarially Optimal Framework for Causal Inference

Estimation of sample average treatment effect is used to illustrate the described method. This is extended to other estimate, such as those for sample average treatment effect of the treated, policy evaluation, and etc. The method is applied to a dataset of N units in the form of [symbol] ={(Xᵢ, Tᵢ, [symbol] where Xᵢ is i-th component of the observed covariates 505, Tᵢ is the i-th component of the observed treatment 510, and Yᵢ is the i-th component of the observed outcome. Suppose Tᵢ∈{0, 1} and let Yᵢ(t) be the potential outcome of assigning treatment Tᵢ=t. The sample average treatment effect is defined as

$${τ}_{SATE} = \frac{1}{N} {\sum }_{i = 1} ( {Y}_{i} ( 1 ) - {Y}_{i} ( 0 ) ) .$$

Assume Yᵢ=Yᵢ(Tᵢ), implying consistency between observed and potential outcomes and non-interference between units and Yᵢ(0), Yᵢ(1)⊥Tᵢ|Xᵢ, implying no latent confounders. Weighted estimators are in the form of

$$\hat{τ} = {\sum }_{i ∈ 𝕋} {α}_{i} {Y}_{i} ( 1 ) - {\sum }_{i ∈ ℂ} {α}_{i} {Y}_{i} ( 0 )$$

where [symbol] ={i∈[N]: Tᵢ=1} is the treated group and [symbol] ={i∈[N]:Tᵢ=0} is the control group. The weighted estimators are calculated using a constrained difference between outcomes of assigning treatments of 0 and 1. Constraints on the weight by are forced allowing α∈ [symbol] m={0 [symbol] α [symbol] 1, [symbol] = [symbol] αᵢ=1}. These constraints help with obtaining robust estimators. For example, [symbol] =1 ensures that the bias remains unchanged if a constant is added to the outcome model of the treated, whereas [symbol] =1 further ensures that the bias remains unchanged if the same constant is added to the outcome model of the control.

A good estimator should minimize the absolute value of the conditional bias that can be written as

$$𝔼 ( \hat{τ} - {τ}_{SATE} \vert {\{ {X}_{i} , {T}_{i} \}}_{i = 1}^{𝒩} ) = {\sum }_{𝒾 = 1}^{N} ( {α}_{i} {T}_{i} - \frac{1}{N} ) 𝔼 ( {Y}_{i} ( 1 ) - {Y}_{i} ( 0 ) \vert {X}_{i} ) + {\sum }_{𝒾 = 1}^{N} {α}_{i} {W}_{i} 𝔼 ( {Y}_{i} ( 0 ) \vert {X}_{i} )$$

where Wᵢ=1 if i∈ [symbol] and Wᵢ=−1 if i∈ [symbol] . In other words, Wᵢ=1 if I is in the treatment group and Wᵢ=−1 if i is in the control group. As the outcome models are typically unknown, previous works are followed by minimizing an upper bound of the square of the second term. Namely, assuming the outcome model E(Yᵢ(0)|Xᵢ) belongs to a hypothesis class [symbol] , [symbol] [symbol] (Σᵢ₌₁^{N}α, Wᵢ∫(Xᵢ))² is solved. To simplify this, consider [symbol] being an unit-ball reproducing kernel Hilbert space (RKHS) defined by some feature map ϕ. Then a supremum can be computed in closed form, which reduces the optimization problem to

$${min}_{α ⊂ 𝒜} {α}^{⊤} {K}_{ϕ} α , \qquad ( 1 )$$

where |K_{ϕ}|=WᵢWⱼϕ(Xᵢ)^{T}φ(Xⱼ). It is recognized herein that Eq. (1) is equivalent to a dual SVM problem, which is described subsequently.

### Causal Inference as Dual SVM and Support Vector Expansions

In order to learn optimal balancing weights via training an attention network, an important insight herein is that Eq. (1) may be re-derived as a dual SVM problem. Suppose a treatment assignment Wᵢ is classified based on feature vector ϕ(Xᵢ) via SVM, by solving the following optimization problem,

$$\begin{matrix}\frac{λ}{2} {‖ β ‖}^{2} + {\sum }_{i = 1}^{N} {ξ}_{i} , \\ \begin{matrix}{W}_{i} ( 〈 β , ϕ ( {X}_{i} ) 〉 + {β}_{0} ) \geq 1 - {ξ}_{i} , & {ξ}_{i} \geq 0 , & ∀ i ∈ [ N ] .\end{matrix}\end{matrix} \qquad ( 2 )$$

Here [symbol] ·,· [symbol] denotes the inner product of the Hilbert space to which ϕ projects. The dual form of this problem corresponds to

$$\begin{matrix}\begin{matrix}{min}_{α} & {α}^{⊤} {K}_{ϕ} α - 2 λ \cdot {1}^{⊤} α\end{matrix} \\ \begin{matrix}s . t . & \begin{matrix}{W}^{⊤} α = 0 , & 0 ⪯ α ⪯ 1.\end{matrix}\end{matrix}\end{matrix} \qquad ( 3 )$$

This is equivalent to solving Eq. (1) for some λ≥0; in other words, the optimal solution α* to Eq. (3) solves Eq. (1). Thus optimal balancing weight can be obtained by solving the dual SVM.

Another useful result is the support vector expansion of an optimal SVM classifier, which connects the primal solution to the dual coefficients α*. By the Karush-Kuhn-Tucker (KKT) condition, the optimal β* that solves Eq. (2) should satisfy β*= [symbol] α*ⱼWⱼϕ(Xⱼ). Thus the optimal classifier will have the following support vector expansion

$$〈 {β}^{*} , ϕ ( {X}_{i} ) 〉 = {\sum }_{j = 1}^{N} {α}_{j}^{*} {W}_{j} \cdot 〈 ϕ ( {X}_{j} ) . ϕ ( {X}_{i} ) 〉 . \qquad ( 4 )$$

Note that the constant intercept is dropped for simplicity. Later, it is described how the self-attention layer can be written in this form.

The causal inference model may, for example, be implemented using a transformer architecture, with a self-attention layer, or other attention-based architecture. Until recently, state of the art performance has been achieved in various applications with relatively mature neural network architectures, such as convolutional neural networks. However, newer architectures, such as “transformers”, are beginning to surpass the performance of more traditional architectures in a range of applications (such as computer vision and natural language processing). Encoder-decoder neural networks, such as transformers, have been developed based solely or primarily on “attention mechanisms”, removing or reducing the need for more complex convolutional and recurrent architectures.

### Self-Attention as Support Vector Expansion

A neural attention function is applied to a query vector q and a set of key-value pairs. Each key-value pair is formed of a key vector kᵢ and a value vector vᵢ, and the set of key-value pairs is denoted {kᵢ, vᵢ}. An attention score for the ith key-value pair with respect to the query vector q is computed as a softmax of a dot product of the query vector with the ith key value, q·kᵢ. An output is computed as a weighted sum of the value vectors, {vᵢ}, weighted by the attention scores.

For example, in a self-attention attention layer of a transformer, query, key and value vectors are all derived from an input sequence (inputted to a self-attention layer) through matrix multiplication. The input sequence comprises multiple input vectors at respective sequence positions, and may be an input to the transformer (e.g., tokenized and embedded text, image, audio etc.) or a ‘hidden’ input from another layer in the transformer. For each input vector xⱼ in the input sequence, a query vector qⱼ, a key vector kⱼ and a value vector vⱼ are computed through matrix multiplication of the input vector xⱼ with learnable matrices W^{Q}, W^{V}, W^{K}. An attention score α_{i,j} for every input vector xᵢ with respect to position j (including i=j) is given by the softmax of qⱼ·kᵢ. An output vector yⱼ for token j is computed as a weighted sum of the values ν₁, ν₂, . . . , weighted by their attention scores: yⱼ=Σᵢr_{i,j}νᵢ. The attention score r_{i,j} captures the relevance (or relative importance) of input vector xⱼ to input vector xᵢ. Whilst the preceding example considers self-attention, similar mechanisms can be used to implement other attention mechanisms in neural networks, such as cross-attention.

The ‘query-key-value’ terminology reflects parallels with a data retrieval mechanism, in which a query is matched with a key to return a corresponding value. As noted above, in traditional neural attention, the query is represented by a single embedding vector q. In this context, an attention layer is, in effect, querying knowledge that is captured implicitly (in a non-interpretable, non-verifiable and non-correctable manner) in the weights of the neural network itself.

Consider input sequence as X=[x₁, . . . , x_{N}]^{T}∈ [symbol] . Here, the transpose of the covariate matrix 505 is of size N by D. A self-attention layer transforms X 505 into an output sequence via

$$softmax ( {QK}^{⊤} / \sqrt{D} ) V ,$$

Where Q=[q₁, . . . , [symbol] ]^{T} ∈ [symbol] , K=[k₁, . . . , k_{N}]^{T} ∈ [symbol] , and V=[r₁, . . . , [symbol] ]^{T} ∈ [symbol] . Here, an output is considered to be a sequence of scalars: in general, V can be a sequence of vectors. Query and key matrices [symbol] , K 501 can be X 505 itself or outputs of several neural network layers on X 505. Note that a softmax operation 570 is with respect to per column of αk^{T}/\sqrt{D}), i.e., the i-th output is

$${{\sum }^{N}}_{j = 1} \frac{exp ( {q}_{i} {k}_{j}^{⊤} / \sqrt{D} )}{{\sum }_{{j}^{′} = 1}^{N} exp ( {q}_{i} {k}_{{j}^{′}}^{⊤} / \sqrt{D} )} {v}_{j} .$$

Here, Q is set equal to K, 501, and therefore there exists a feature map such that for any i, j∈[N](ϕ(Xⱼ), ϕ(Xᵢ [symbol] =exp (k,kⱼ^{T}/\sqrt{D})¹. Let h(Xᵢ)=Σⱼ^{N} exp(k,kⱼ^{T}/\sqrt{D}). Let h(Xᵢ)= [symbol] exp (k,kⱼ^{T}/\sqrt{D}).

The i-th output of attention layer can be re-written as

$${{\sum }^{N}}_{j = 1} \frac{{v}_{j}}{h ( {X}_{j} )} 〈 ϕ ( {X}_{j} ) , ϕ ( {X}_{i} ) 〉 . \qquad ( 5 )$$

This formulation recovers the support vector expansion in Eq. (4) upon optimal solution given by the KKT condition, νⱼ/h(Xᵢ)=αⱼWⱼ.

Conversely, under mild regularities, the optimal balancing weight α*j 580 can be read from νⱼ/h(Xⱼ)Wⱼ if the attention weight is optimised globally using a crafted loss function. The details are presented in Algorithm 1. The intuition is that this loss function, when optimized globally, recovers attention weights that solve the primal SVM problem. Thus it recovers the support vector expansion, which connects the attention weight to the optimal balancing weight 580. The correctness of this algorithm is summarised in the following theorem.

Theorem 1 Under mild regularities on X, Algorithm 1 recovers the optimal balancing weight at the global minimum of LOSS FUNC.

### Practical Algorithm Towards Causal Transformers

The former derivations can be used to obtain an algorithm that provably achieves optimal balancing on one dataset. The resulting model can be optimized via gradient descent methods, allowing for incorporation of flexible neural network architectures. It is subsequently shown how it can be amortized, which permits generalizing to new datasets unseen during training. A new type of data augmentation for the amortized model is also proposed. Finally it is shown how the trained model can be used to estimate counterfactuals, thereby addressing various causal inference tasks.

### Optimal Balancing Via Self-Attention

Comparing Eq. (5) and Eq. (4), a training procedure is needed such that

$${\sum }_{j = 1}^{N} \frac{\text{?}}{\text{?} ( {X}_{j} )} ϕ ( {X}_{j} )$$

$$\text{?} \text{indicates text missing or illegible when filed}$$

recovers the optimal β* that solves primal SVM in Eq. (2). Note that Eq. (2) corresponds to a constrained optimization problem that is unsuitable for gradient descent methods. However, it is equivalent to an unconstrained optimization problem by minimizing the penalized hinge loss

$$\frac{\text{?}}{2} {‖ β ‖}^{2} + {\sum }_{\text{?} = 1}^{\text{?}} [ 1 - {W}_{i} ( 〈 β , ϕ ( {X}_{i} ) 〉 + {β}_{0} ) ] \text{?} .$$

$$\text{?} \text{indicates text missing or illegible when filed}$$

This motivates the use of the following loss function:

$${ℒ}_{θ} = \frac{λ}{2} {‖ {{\sum }^{N}}_{j = 1} \frac{{v}_{j}}{h ( {X}_{j} )} ϕ ( {X}_{j} ) ‖}^{2} + [ 1 - W ( softmax ( {KK}^{⊤} / \sqrt{D} ) V + {β}_{0} ) ] \text{?} . \qquad ( 6 )$$

$$\text{?} \text{indicates text missing or illegible when filed}$$

Here θ is used to subsume all learned parameters, including V and parameters of the layers (if any) to obtain K 501. θ is learned via gradient descent on Eq. (6). Note that the penalization can be computed exactly by using the formula of inner product of features, i.e.,

$${‖ {{\sum }^{N}}_{j = 1} \frac{{v}_{j}}{h ( {X}_{j} )} ϕ ( {X}_{j} ) ‖}^{2} = {{\sum }^{N}}_{\text{?} j = 1} \frac{{v}_{i} {v}_{j} exp ( {k}_{i} {k}_{j}^{⊤} / \sqrt{D} )}{h ( {X}_{i} ) h ( {X}_{j} )} .$$

$$\text{?} \text{indicates text missing or illegible when filed}$$

Theorem 1 guarantees that under mild regularities, the optimal parameters lead to the optimal balancing weights 580 in terms of the adversarial squared error. This adversarial squared error is computed using an unit-ball RKHS defined by ϕ. The optimal balancing weights 580 can be obtained via

$${α}_{j}^{*} = \frac{{v}_{j}}{h ( {X}_{j} ) {W}_{j}} .$$

Note that for this result to hold, arbitrary mapping can be used to obtain Kᵢ 501 from Xᵢ 505, thus allowing for the incorporation of flexible neural network architecture. This method is summarized in Algorithm 1.

ALGORITHM 1
CINA.

| | |
|---|---|
| 1: | Input: Covariates X and treatment W. |
| 2: | Output: Optimal balancing weight α*. |
| 3: | Parameters: θ (including V), step size η. |
| 4: | while not converged do |
| 5: | Compute K using forward pass. |
| 6: | Update θ ← θ − η∇ [symbol] . |
| 7: | return V /h(X)W. |

### Inference on Multiple Datasets

To enable direct inference of treatment effects, multiple datasets denoted [symbol] ={(Xᵢ, Tᵢ, [symbol] as for m∈[M] are considered. Each dataset Dₘ contains Nₘ units as described above. The method allows for datasets of different sizes, mimicking real-world data gathering procedures, where a large consortium of datasets in a similar format may exist. The setting encapsulates cases where individual datasets are created by distinct causal mechanisms or rules; however, different units within a single dataset should be generated via the same causal model.

Algorithm 1 determines the components used to calculate optimal weights α* 580 from a trained model with attention as its last layer in a single dataset. Note that the value vector V 565 is encoded as a set of parameters in this setting. On a new dataset, the values of h(X) and W are changed, and thus the optimal V that minimizes L_{θ} should also differ from the encoded parameters. To account for this, the value vector V is encoded as a transformation of h(X) and W. The parameters of this transformation are denoted as ϕ. ϕ is learned by minimizing L_{θ} on the training datasets in an end-to-end fashion. Then on a new dataset not seen during training, its optimal balancing weight α* can be directly inferred via V/h(X) W where V and h(X) are direct output using the forward pass of the trained model. This procedure is summarized in Algorithm 2 and Algorithm 3.

ALGORITHM 2
CINA (multi-dataset version).

| | |
|---|---|
| 1: | Input: Training datasets [symbol] , ..., [symbol] . |
| 2: | Parameters: θ (including ϕ), step size η. |
| 3: | while not converged do |
| 4: | for m ϵ [M] do |
| 5: | Compute K, V using forward pass. |
| 6: | Update θ ← θ − η∇ [symbol] . |

ALGORITHM 3
Inference.

| | |
|---|---|
| 1: | Input: Testing dataset [symbol] , trained model. |
| 2: | Output: Estimated sample average treatment |
| | effect \hat{T}. |
| 3: | Compute h(X), V using forward pass. |
| 4: | Compute α = V /h(X)W. |
| 5: | return αY. |

In an extension, and additional penalty weight λ>0 hyperparameter may be included, with α*=λ·V^{(*)}/h(X^{(*)})W^{(*)}.

Intuitively, the transformation that encodes for V approximates the solution to the optimization problem in Eq. (2). It enjoys the benefit of fast inference on a new dataset. It is worth noting that it does not require ground-truth labels to any individual optimization problems as the parameters are learned fully end-to-end. This reduces the computational burden of learning in multiple steps, albeit unavoidable trade-off in terms of accuracy.

FIG. 5B shows the inference time for the causal inference model described with reference to FIG. 5A. In this example embodiment, covariates 505, treatments 510, and outcomes 515 relating to a new medicine dataset are considered. However, it will be appreciate the description applies equally to any application or domain.

In this example embodiment, the covariates 505 relate to patients, the treatments 510 relate to new medicine and the outcomes 515 relate to recovery. The causal inference model discussed in relation to FIG. 5A receives the example covariates 505 and treatments 510 as input. When in test mode, the model outputs a rebalancing weight α*, 580. The rebalancing weight 580 and the outcomes 515 are used to determine the effect of the new medicine 590, in this example embodiment. The effect 590 is determined by calculating the sum of the product of the rebalancing weight α*, 580, the outcomes 515 and the treatments 510.

### Supervised Training of CInA

In some implementations, datasets have different average treatment effects (ATEs) from each other may be used. In this case, it is possible to use the ground truth ATEs from the training datasets to serve as additional supervised signal in train. This can be done via simultaneously minimizing Σ_{m∈[M]}∥(V^{(m)}/h(X^{(m)}))^{T}Y^{(m)}−T^{(m)}∥². The new loss hence becomes

$${\sum }_{m ∈ [ M ]} {ℒ}_{θ} ( {𝔻}^{( m )} ) + η {\sum }_{m ∈ [ M ]} {‖ {( {V}^{( m )} / h ( {X}^{( m )} ) )}^{⊤} {Y}^{( m )} - {τ}^{( m )} ‖}^{2} .$$

where η is an adjustable coefficient with default value 1. This is a supervised variation of the above method.

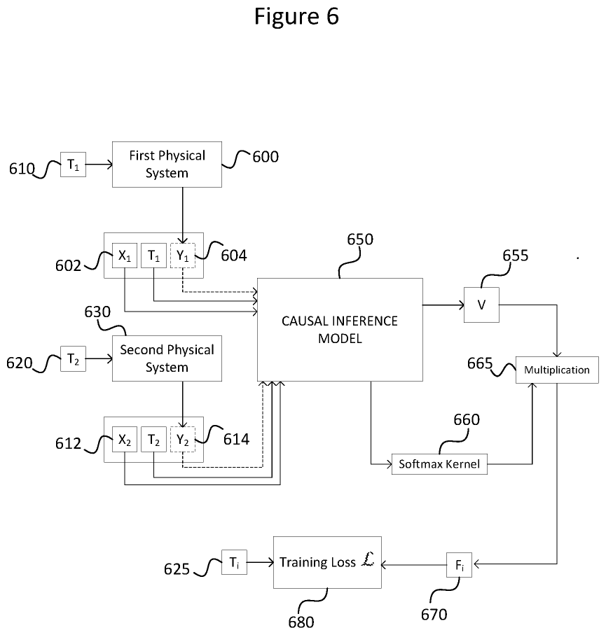

FIG. 6 shows an embodiment of the training method of a causal inference model 650 presented in FIG. 3, with reference to a physical system. A first treatment action 610 is performed on a first physical system 600 to obtain a first outcome 604. Similarly, a second treatment action 620 is performed on a second physical system 630 to obtain a second outcome 614. A first covariate vector 602, a second covariate vector 612 and the first and second treatment vectors 610, 620 are input into a causal inference model 650. The first and second outcomes 604, 614 can also be optionally input into the model 650. The causal inference model 650 outputs a value vector 655. A matrix multiplication 665 is performed between a softmax-kernel 660 and the value vector 655. The output of the matrix multiplication 665 is a simulated forward mode output 670. The simulated forward mode output 670 and a treatment vector 625 are input into a training loss function 680. The training loss function 680 minimises the error between the simulated forward mode output 670 and the treatment vector 625. Training is an interactive process, in which forward mode outputs are computed repeatedly. Initially, the untrained model is applied to compute initial outputs, and the training loss is used to tune model parameters based on the initial outputs (for example, based a gradient of the training loss with respect to each model parameter, where the gradient is evaluated for the initial outputs). This process of computing forward mode outputs and using those outputs to tune the parameters is repeated until some termination condition is reached.

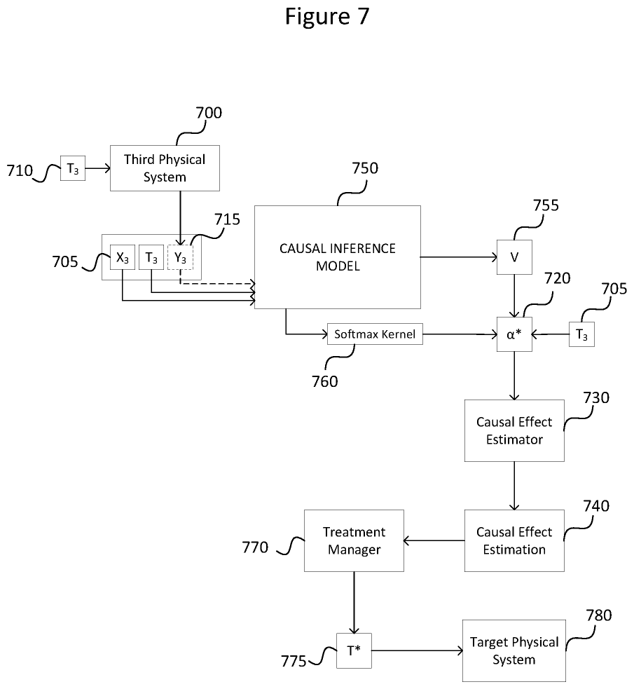

FIG. 7 shows an example causal inference model 750 generating a rebalancing weight vector 720 to determine a further treatment action 775 to be performed on a target physical system 780. A third treatment action 710 is performed on a third physical system 700 to generate a third outcome 715. A third covariate vector 705 and the third treatment action 710 is input into the causal inference model 750. The third outcome 715 can optionally be input into the model 750. The causal inference model 750 outputs a value vector 755. A softmax-kernel 760, the value vector 755 and the third treatment action 710 are used to generate a rebalancing weight 720. The rebalancing weight 720 is input into a causal effect estimator 730 to generate a causal effect estimation 740. The causal effect estimation 740 is input into a treatment manager 770. The treatment manager 770 selects the further treatment action 775 to be performed on the target physical system 780.

The causal inference technology described herein can be applied to novel scenarios whenever causal effects needs to be identified. For example: i) In manufacturing industry, it may be desirable to quantitatively identify the impact from different factors that affect product quality, production efficiency, and machinery performance in manufacturing processes. Given a quantitative causal model and certain amount of trial data, the method would allow better and faster understanding of how well this model can predict certain the causal relationships between these factors, companies can optimize their processes, reduce waste, and improve overall efficiency; ii), in aerospace industry, root cause analysis is crucial to identify the underlying causes of faults and malfunctions in aircraft systems. The method can help evaluating which root causal analysis method is the most efficient for guiding targeted maintenance and repair actions, by analyzing experimental data from sensors, maintenance records, and incident reports, causal inference methods iii) In genome-wide association studies (GWAS), it is important to test a hypothesis that associates between genetic variants and the trait or diseases. The method would accelerate the process of validating those assumptions via experimental data.

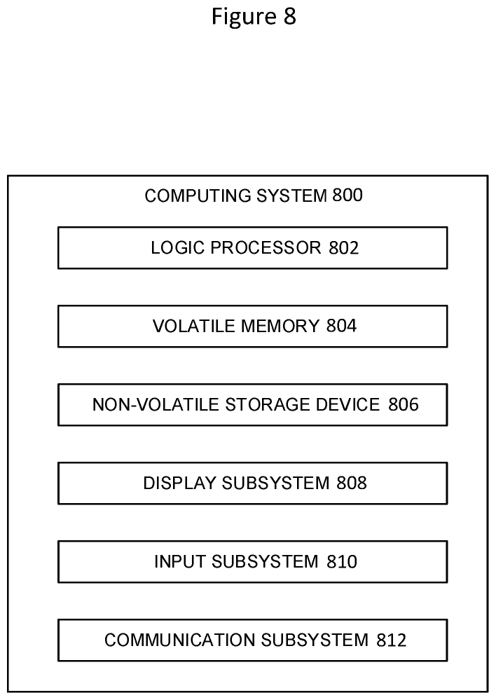

FIG. 8 schematically shows a non-limiting example of a computing system 800, such as a computing device or system of connected computing devices, that can enact one or more of the methods or processes described above, including the filtering of data and implementation of the structured knowledge base described above. Computing system 800 is shown in simplified form. Computing system 800 includes a logic processor 802, volatile memory 804, and a non-volatile storage device 806. Computing system 800 may optionally include a display subsystem 808, input subsystem 810, communication subsystem 812, and/or other components not shown in FIG. 8. Logic processor 802 comprises one or more physical (hardware) processors configured to carry out processing operations. For example, the logic processor 802 may be configured to execute instructions that are part of one or more applications, programs, routines, libraries, objects, components, data structures, or other logical constructs. The logic processor 802 may include one or more hardware processors configured to execute software instructions based on an instruction set architecture, such as a central processing unit (CPU), graphical processing unit (GPU) or other form of accelerator processor. Additionally or alternatively, the logic processor 802 may include a hardware processor(s)) in the form of a logic circuit or firmware device configured to execute hardware-implemented logic (programmable or non-programmable) or firmware instructions. Processor(s) of the logic processor 802 may be single-core or multi-core, and the instructions executed thereon may be configured for sequential, parallel, and/or distributed processing. Individual components of the logic processor optionally may be distributed among two or more separate devices, which may be remotely located and/or configured for coordinated processing. Aspects of the logic processor 802 may be virtualized and executed by remotely accessible, networked computing devices configured in a cloud-computing configuration. In such a case, these virtualized aspects are run on different physical logic processors of various different machines. Non-volatile storage device 806 includes one or more physical devices configured to hold instructions executable by the logic processor 802 to implement the methods and processes described herein. When such methods and processes are implemented, the state of non-volatile storage device 806 may be transformed—e.g., to hold different data. Non-volatile storage device 806 may include physical devices that are removable and/or built-in. Non-volatile storage device 806 may include optical memory (e g., CD, DVD, HD-DVD, Blu-Ray Disc, etc.), semiconductor memory (e g., ROM, EPROM, EEPROM, FLASH memory, etc.), and/or magnetic memory (e.g., hard-disk drive), or other mass storage device technology. Non-volatile storage device 806 may include nonvolatile, dynamic, static, read/write, read-only, sequential-access, location-addressable, file-addressable, and/or content-addressable devices. Volatile memory 804 may include one or more physical devices that include random access memory. Volatile memory 804 is typically utilized by logic processor 802 to temporarily store information during processing of software instructions. Aspects of logic processor 802, volatile memory 804, and non-volatile storage device 806 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example. The terms “module,” “program,” and “engine” may be used to describe an aspect of computing system 800 typically implemented in software by a processor to perform a particular function using portions of volatile memory, which function involves transformative processing that specially configures the processor to perform the function. Thus, a module, program, or engine may be instantiated via logic processor 802 executing instructions held by non-volatile storage device 806, using portions of volatile memory 804. Different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc. When included, display subsystem 808 may be used to present a visual representation of data held by non-volatile storage device 806. The visual representation may take the form of a graphical user interface (GUI). As the herein-described methods and processes change the data held by the non-volatile storage device, and thus transform the state of the non-volatile storage device, the state of display subsystem 808 may likewise be transformed to visually represent changes in the underlying data. Display subsystem 808 may include one or more display devices utilizing virtually any type of technology. Such display devices may be combined with logic processor 802, volatile memory 804, and/or non-volatile storage device 806 in a shared enclosure, or such display devices may be peripheral display devices. When included, input subsystem 810 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity; and/or any other suitable sensor. When included, communication subsystem 812 may be configured to communicatively couple various computing devices described herein with each other, and with other devices. Communication subsystem 812 may include wired and/or wireless communication devices compatible with one or more different communication protocols. As non-limiting examples, the communication subsystem may be configured for communication via a wireless telephone network, or a wired or wireless local- or wide-area network. In some embodiments, the communication subsystem may allow computing system 800 to send and/or receive messages to and/or from other devices via a network such as the internet. The term computer readable media as used herein may include computer storage media. Computer storage media may include volatile and non-volatile, removable and nonremovable media (e.g., volatile memory 804 or non-volatile storage 806) implemented in any method or technology for storage of information, such as computer readable instructions, data structures, or program modules. Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information, and which can be accessed by a computing device (e.g. the computing system 800 or a component device thereof). Computer storage media does not include a carrier wave or other propagated or modulated data signal. Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct wired connection, and wireless media such as acoustic, radio frequency (RF), infrared, and other wireless media.

It will be appreciated that the above embodiments have been disclosed by way of example only. Other variants or use cases may become apparent to a person skilled in the art once given the disclosure herein. The scope of the present disclosure is not limited by the above-described embodiments, but only by the accompanying claim.

Features of the disclosure are defined in the statements below.

A first aspect of the present disclosure provides a computer-implemented method, comprising: receiving a first training dataset specific to a first domain, the first training dataset comprising a first covariate matrix characterizing a first system and a first treatment vector encoding a first treatment observation relating to the first system; receiving a second training dataset specific to a second domain, the second training dataset comprising a second covariate matrix characterizing a second system and a second treatment vector encoding a second treatment observation relating to the second system; computing using a causal inference model applied to the first training dataset a first forward mode output corresponding to the first treatment vector; computing using the causal inference model applied to the second training dataset a second forward mode output corresponding to the second treatment vector; training the causal inference model based on a training loss that quantifies error between: the first treatment vector and the first forward mode output, and the second treatment vector and the second forward mode output, resulting in a trained causal inference model; computing a rebalancing weight vector using the trained causal inference model applied to a third dataset specific to a third domain, the third dataset comprising a third covariate matrix characterizing a third system, a third treatment vector encoding a third treatment observation and a third outcome vector; estimating based on the third outcome vector and the rebalancing weight vector a causal effect associated with the third treatment vector; based on the causal effect, determining a treatment action; and performing the treatment action on at least one target system belonging to the third domain

In embodiments, the third dataset may be specific to a third domain, wherein the causal inference model may not be exposed to any data from the third domain during training.

The first training dataset, the second training dataset and the third dataset may each be non-randomized.

The at least one third system may comprise the at least one target system.

The at least one target system may comprise a machine and the causal effect may comprise an estimated treatment effect pertaining to performance of the machine.

The machine may be a manufacturing machine, and the estimated treatment effect may pertain to: quality of a product manufactured using the machine, or production efficiency of the machine.

The at least one target system may comprise a computer system and the causal effect may comprise an estimated treatment effect pertaining to usage of memory or processing resources.

The causal inference model may generate during training: a first output value, wherein the forward mode output corresponding to the first training dataset may be computed based on the first output value and a first normalization factor may be computed from the first covariate matrix, and a second output value, wherein the forward mode output may correspond to the second training dataset which may be computed based on the second output value and a second normalization factor may be computed from the first second matrix; wherein the rebalancing weight vector may be computed based on: a third output value which may be computed by the trained causal inference model, the third treatment vector, and a third renormalization factor which may be computed from the third covariate matrix.

The causal effect may be determined based on a summation of a product of: the rebalancing weight vector, the third treatment vector and the third outcome vector.

The causal inference model may have a transformer neural network architecture.

A second aspect of the present disclosure provides a computer system comprising: at least one memory configured to store computer-readable instructions; and at least one hardware processor coupled to the at least one memory, wherein the computer-readable instructions are configured to cause the at least one hardware processor to implement operations comprising: receiving a first training dataset specific to a first domain, the first training dataset comprising a first covariate matrix characterizing a first system and a first treatment vector encoding a first treatment observation relating to the first system; receiving a second training dataset specific to a second domain, the second training dataset comprising a second covariate matrix characterizing a second system and a second treatment vector encoding a second treatment observation relating to the second system; computing using a causal inference model applied to the first training dataset a first forward mode output corresponding to the first treatment vector; computing using the causal inference model applied to the second training dataset a second forward mode output corresponding to the second treatment vector; training the causal inference model based on a training loss that quantifies error between: the first treatment vector and the first forward mode output, and the second treatment vector and the second forward mode output, resulting in a trained causal inference model; computing a rebalancing weight vector using the trained causal inference model applied to a third dataset specific to a third domain, the third dataset comprising a third covariate matrix characterizing a third system, a third treatment vector encoding a third treatment observation relating to the third system and a third outcome vector; estimating based on the third outcome vector and the rebalancing weight vector a causal effect associated with the third treatment vector; based on the causal effect, determining a treatment action.

In embodiments, said operations may comprise automatically performing the treatment action on at least one target system belonging to the third domain.

The at least one third system may comprise the at least one target system.

The third dataset may be specific to a third domain, wherein the causal inference model may not be exposed to any data from the third domain during training.

The first training dataset, the second training dataset and the third dataset may each be non-randomized.

The causal effect may comprise an estimated treatment effect pertaining to performance of a machine.

The machine may be a manufacturing machine, and the estimated treatment effect may pertain to quality of a product manufactured using the machine, or production efficiency of the machine.

The causal effect may comprise an estimated treatment effect pertaining to usage of memory or processing resources by a computer system.

The causal inference model may have a transformer neural network architecture.

Further optional features of the second aspect are as defined above in relation to the first aspect and may be combined in any combination.

A third aspect of the present disclosure provides a computer-readable storage media embodying computer readable instructions, the computer-readable instructions configured upon execution on at least one hardware processor to cause the at least one hardware processor to implement operations comprising: computing a rebalancing weight vector using a trained causal inference model applied to a third dataset specific to a third domain, the trained causal inference model having been trained by: receiving a first training dataset specific to a first domain, the first training dataset comprising a first covariate matrix characterizing a first system and a first treatment vector encoding a first treatment observation relating to the first system, receiving a second training dataset specific to a second domain, the second training dataset comprising a second covariate matrix characterizing a second system and a second treatment vector encoding a second treatment observation relating to the second system, and computing using a causal inference model applied to the first training dataset a first forward mode output corresponding to the first treatment vector; computing using the causal inference model applied to the second training dataset a second forward mode output corresponding to the second treatment vector; training the causal inference model based on a training loss that quantifies error between: the first treatment vector and the first forward mode output, and the second treatment vector and the second forward mode output, resulting in a trained causal inference model; the third dataset comprising a third covariate matrix characterizing a third system, a third treatment vector encoding a third treatment observation of the third system and a third outcome vector; estimating based on the third outcome vector and the rebalancing weight vector a causal effect associated with the third treatment vector: based on the causal effect, determining a treatment action.

Further optional features of the third aspect are as defined in relation to the first and second aspect and may be combined in any combination.

1. A computer-implemented method, comprising:
receiving a first training dataset specific to a first domain, the first training dataset comprising a first covariate matrix characterizing a first system and a first treatment vector encoding a first treatment observation relating to the first system;
receiving a second training dataset specific to a second domain, the second training dataset comprising a second covariate matrix characterizing a second system and a second treatment vector encoding a second treatment observation relating to the second system;
computing using a causal inference model applied to the first training dataset a first forward mode output corresponding to the first treatment vector;
computing using the causal inference model applied to the second training dataset a second forward mode output corresponding to the second treatment vector;
training the causal inference model based on a training loss that quantifies error between:
the first treatment vector and the first forward mode output, and
the second treatment vector and the second forward mode output, resulting in a trained causal inference model:
computing a rebalancing weight vector using the trained causal inference model applied to a third dataset specific to a third domain, the third dataset comprising a third covariate matrix characterizing a third system, a third treatment vector encoding a third treatment observation and a third outcome vector;
estimating based on the third outcome vector and the rebalancing weight vector a causal effect associated with the third treatment vector;
based on the causal effect, determining a treatment action; and
performing the treatment action on at least one target system belonging to the third domain.

2. The method of claim 1, wherein the third dataset is specific to a third domain, wherein the causal inference model is not exposed to any data from the third domain during training.

3. The method of claim 1, wherein the first training dataset, the second training dataset and the third dataset are each non-randomized.

4. The method of claim 1, wherein the at least one third system comprises the at least one target system.

5. The method of claim 1, wherein the at least one target system comprises a machine and the causal effect comprises an estimated treatment effect pertaining to performance of the machine.

6. The method of claim 5, wherein the machine is a manufacturing machine, and the estimated treatment effect pertains to:
quality of a product manufactured using the machine, or
production efficiency of the machine.

7. The method of claim 1, wherein the at least one target system comprises a computer system and the causal effect comprises an estimated treatment effect pertaining to usage of memory or processing resources.

8. The method of claim 1, wherein the causal inference model generates during training:
a first output value, wherein the forward mode output corresponding to the first training dataset is computed based on the first output value and a first normalization factor computed from the first covariate matrix, and
a second output value, wherein the forward mode output corresponding to the second training dataset is computed based on the second output value and a second normalization factor computed from the second covariate matrix;
wherein the rebalancing weight vector is computed based on: a third output value computed by the trained causal inference model, the third treatment vector, and a third renormalization factor computed from the third covariate matrix.

9. The method of claim 1, wherein the causal effect is determined based on a summation of a product of: the rebalancing weight vector, the third treatment vector and the third outcome vector.

10. The method of claim 1, wherein the causal inference model has a transformer neural network architecture.

11. A computer system comprising:
at least one memory configured to store computer-readable instructions; and
at least one hardware processor coupled to the at least one memory, wherein the computer-readable instructions are configured to cause the at least one hardware processor to implement operations comprising:
receiving a first training dataset specific to a first domain, the first training dataset comprising a first covariate matrix characterizing a first system and a first treatment vector encoding a first treatment observation relating to the first system;
receiving a second training dataset specific to a second domain, the second training dataset comprising a second covariate matrix characterizing a second system and a second treatment vector encoding a second treatment observation relating to the second system;
computing using a causal inference model applied to the first training dataset a first forward mode output corresponding to the first treatment vector;
computing using the causal inference model applied to the second training dataset a second forward mode output corresponding to the second treatment vector;
training the causal inference model based on a training loss that quantifies error between:
the first treatment vector and the first forward mode output, and
the second treatment vector and the second forward mode output, resulting in a trained causal inference model:
computing a rebalancing weight vector using the trained causal inference model applied to a third dataset specific to a third domain, the third dataset comprising a third covariate matrix characterizing a third system, a third treatment vector encoding a third treatment observation relating to the third system and a third outcome vector;
estimating based on the third outcome vector and the rebalancing weight vector a causal effect associated with the third treatment vector;
based on the causal effect, determining a treatment action.

12. The computer system of claim 11, wherein said operations comprise:
automatically performing the treatment action on at least one target system belonging to the third domain.

13. The computer system of claim 11, wherein the at least one third system comprises the at least one target system.

14. The computer system of claim 11, wherein the third dataset is specific to a third domain, wherein the causal inference model is not exposed to any data from the third domain during training.

15. The computer system of claim 11, wherein the first training dataset, the second training dataset and the third dataset are each non-randomized.

16. The computer system of claim 11, wherein the causal effect comprises an estimated treatment effect pertaining to performance of a machine.

17. The computer system of claim 16, wherein the machine is a manufacturing machine, and the estimated treatment effect pertains to:
quality of a product manufactured using the machine, or
production efficiency of the machine.

18. The computer system of claim 16, wherein the causal effect comprises an estimated treatment effect pertaining to usage of memory or processing resources by a computer system.

19. The computer system of claim 11, wherein the causal inference model has a transformer neural network architecture.

20. Computer-readable storage media embodying computer readable instructions, the computer-readable instructions configured upon execution on at least one hardware processor to cause the at least one hardware processor to implement operations comprising:
computing a rebalancing weight vector using a trained causal inference model applied to a third dataset specific to a third domain, the trained causal inference model having been trained by:
receiving a first training dataset specific to a first domain, the first training dataset comprising a first covariate matrix characterizing a first system and a first treatment vector encoding a first treatment observation relating to the first system,
receiving a second training dataset specific to a second domain, the second training dataset comprising a second covariate matrix characterizing a second system and a second treatment vector encoding a second treatment observation relating to the second system, and
computing using a causal inference model applied to the first training dataset a first forward mode output corresponding to the first treatment vector;
computing using the causal inference model applied to the second training dataset a second forward mode output corresponding to the second treatment vector;
training the causal inference model based on a training loss that quantifies error between:
the first treatment vector and the first forward mode output, and
the second treatment vector and the second forward mode output,
resulting in a trained causal inference model;
the third dataset comprising a third covariate matrix characterizing a third system, a third treatment vector encoding a third treatment observation of the third system and a third outcome vector;
estimating based on the third outcome vector and the rebalancing weight vector a causal effect associated with the third treatment vector;
based on the causal effect, determining a treatment action.
